(19) United States
(12) Patent Application Publication
(10) Pub. No.: US 2007/0055161 A1
(43) Pub. Date: Mar. 8, 2007

(54) Ultrasonic imaging system and method for simulataneous display of blood flow and perfusion parameters

(75) Inventors: Rohit Garg, Bothell, WA (US); Damien Dolimier, Salem, MA (US); Danny Skyba, Bothell, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 10/578,632

(22) PCT Filed: Nov. 22, 2004
(86) PCT No.: PCT/IB04/52505

§ 371 Date: May 9, 2006

Related U.S. Application Data
(60) Provisional application No. 60/526,535, filed on Dec. 3, 2003.

(51) Int. Cl. A61B 8/14

(52) U.S. Cl. 600/458

(57) ABSTRACT

A method and system are described for displaying an ultrasonic parametric image showing tissue perfusion in registration with an anatomical ultrasonic image of the tissue containing the blood flow. The relative opacities of the parametric image and the anatomical image can be varied, enabling the clinician to view both the perfusion parameters and the blood flow simultaneously or in rapid succession. In an illustrated embodiment the anatomical image or the parametric image can be viewed alone, or in anatomical registration with different or equal opacities. The relative opacity can be changed in a smoothly continuous or stepwise manner.

This is a continuation in part application of U.S. patent application Ser. No. 10/025,200, filed Dec. 18, 2001.

This invention relates to diagnostic ultrasonic imaging, and, more particularly, to a system and method for simultaneously displaying blood flow and tissue perfusion parameters.

Ultrasonic diagnostic imaging systems are capable of imaging and measuring the physiology within the body in a completely noninvasive manner. Ultrasonic waves are transmitted into the body from the surface of the skin and are reflected from tissue and cells within the body. The reflected echoes are received by an ultrasonic transducer and processed to produce an image or measurement of blood flow. Diagnosis is thereby possible with no invasion of the body of the patient.

Materials known as ultrasonic contrast agents can be introduced into the body to enhance ultrasonic diagnosis. Contrast agents are substances that strongly reflect ultrasonic waves, returning echoes which may be clearly distinguished from those returned by blood and tissue. One class of substances which has been found to be especially useful as an ultrasonic contrast agent is gases, in the form of tiny bubbles called microbubbles. Microbubbles strongly backscatter ultrasound in the body, thereby allowing tissues and blood containing the microbubbles to be readily detectable through special ultrasonic processing. Microbubble contrast agents can be used for imaging the body's vascularized tissues, such as the walls of the heart, since the contrast agent can be injected into the bloodstream and will pass through veins, arteries and capillaries with the blood supply until filtered from the blood stream in the lungs, kidneys and liver.

A diagnostic procedure which is greatly aided by contrast agents is the visualization and measurement of tissue perfusion such as the perfusion of the myocardium with oxygenated blood flow. Perfusion imaging and measurement of perfusion at a designated point in the body is described in U.S. Pat. No. 5,833,613, for instance. The parent application serial number 10,025,200 describes a method and apparatus for making and displaying the results of perfusion measurements for a large region of tissue rather than just a particular sample volume location. Such a capability enables the rapid diagnosis of the perfusion rate of a significant region of tissue such as the myocardium, enabling the clinician to quickly identify small regions of tissue where perfusion is problematic due to ischemia or other bloodflow conditions.

As described in the parent application, tissue perfusion of a two or three dimensional region of the body can be displayed as a parametric overlay of the anatomy being diagnosed. Examples are given in the parent application of an overlay of colors or brightnesses representing different quantified values of perfusion which is displayed over the myocardium. The colors of the color overlay indicate the perfusion of the underlying tissue, with each color corresponding to a different perfusion rate or level. Such a perfusion image is similar in concept to a color flow image, in which a color overlay of blood velocity is shown over the organ or vessel in which the velocity of blood flow is being measured. Like the color flow image, the perfusion overlay does not depict the blood itself, but a parameter of the blood flow, in this case, the perfusion of the underlying tissue.

In such a perfusion image, however, the perfusion overlay obscures the underlying image of the blood flow. The clinician may desire to view both the perfusion parameters and the blood flow in the tissue, but generally this can only be done by viewing either the tissue and blood flow image or the parametric perfusion image separately; the clinician only has the choice of viewing one image or the other. It would be desirable to be able to view both the blood flow and the perfusion parameters simultaneously. It would further be desirable to display the simultaneous images in registration so that the clinician may immediately see and understand both the perfusion in a certain region of interest and the blood flow in the region.

In accordance with the principles of the present invention a method and system display in anatomical registration both a parametric image of tissue perfusion and the blood flow in the tissue. An opacity control enables the user to vary the relative opacity of the blood flow image and the parametric image. In an illustrated embodiment the opacity of both images can be varied continuously, enabling the clinician to simultaneously view the perfusion parameter in a region of interest and the blood flow in that region. The opacity can be varied between a display of only the blood flow image to a display of only the parametric image, as well as intermediate views of both. The relative opacity can be varied continuously or stepwise in discrete levels.

In the drawings:

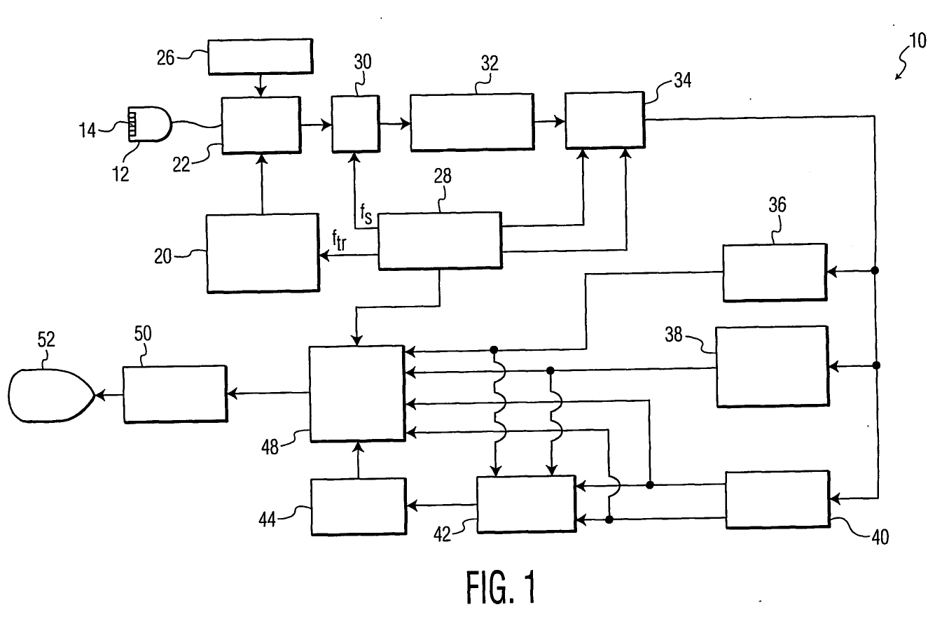

FIG. 1 is a block diagram of an ultrasonic imaging system according to one embodiment of the invention.

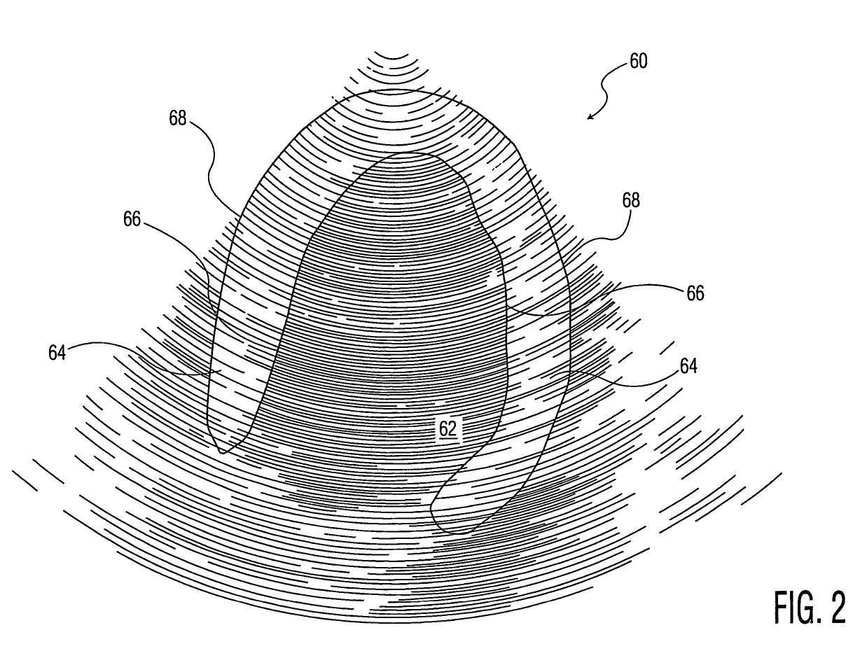

FIG. 2 is a schematic drawing showing a B-mode image of a myocardium obtained using the system of FIG. 1.

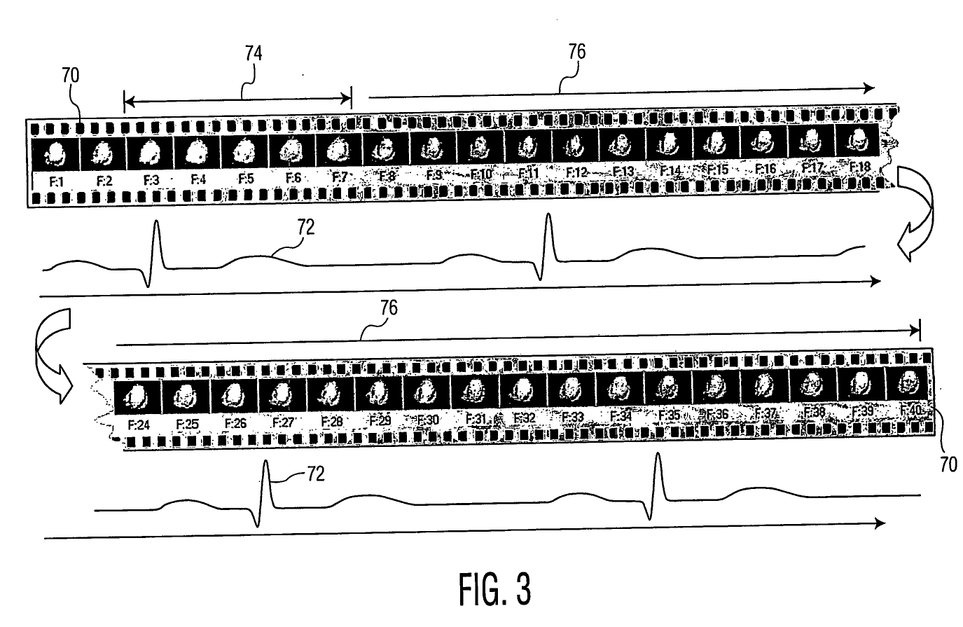

FIG. 3 illustrates the acquisition of a sequence of real time image frames for parametric imaging.

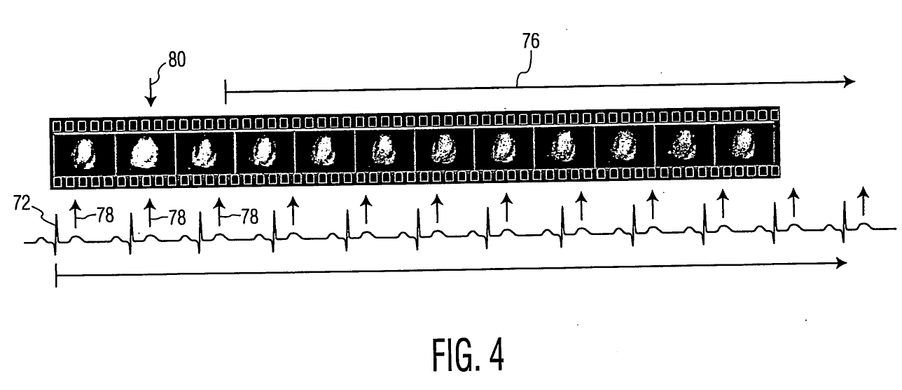

FIG. 4 illustrates gated (triggered) acquisition of a sequence of frames for parametric imaging.

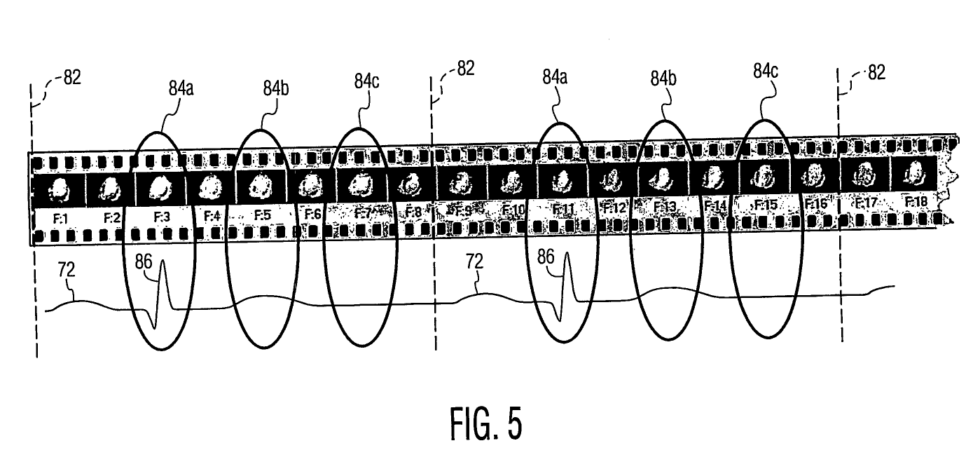

FIG. 5 depicts a sequence of real time images over several heart cycles.

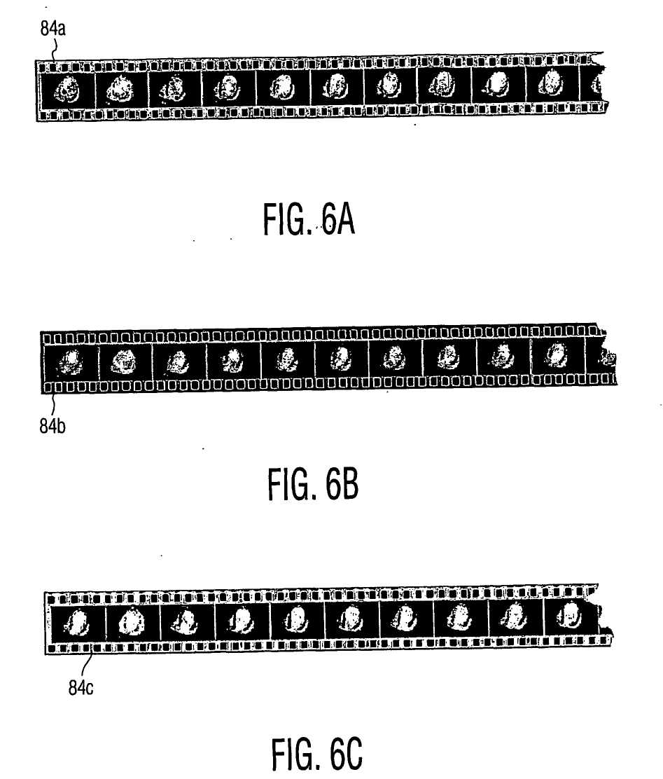

FIGS. 6a, 6b, and 6c illustrate sequences of images for unique phases of the heart cycle assembled from the images of the sequence of FIG. 5.

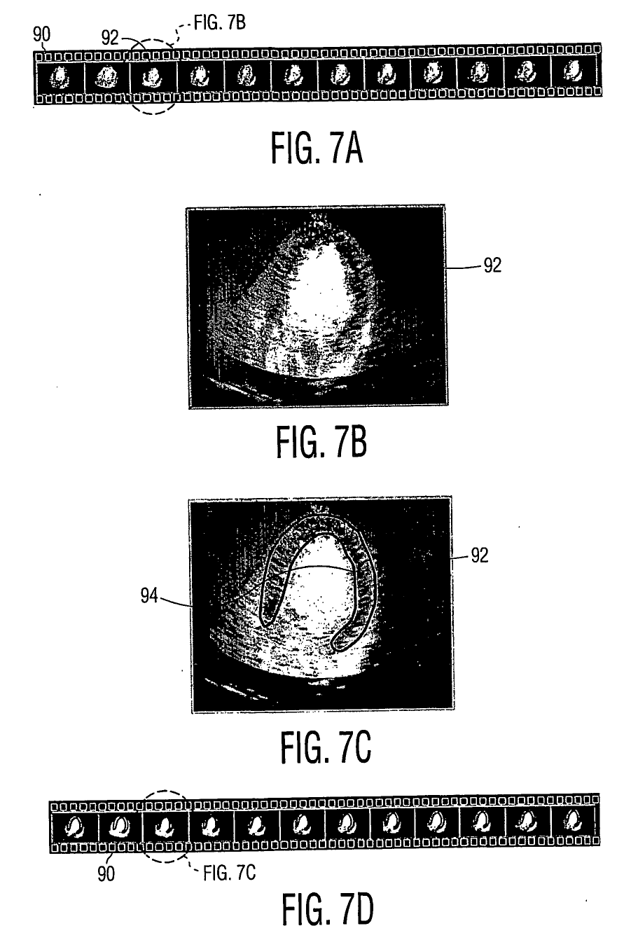

FIGS. 7a-7d illustrate the delineation of a region of interest in an image using assisted border detection.

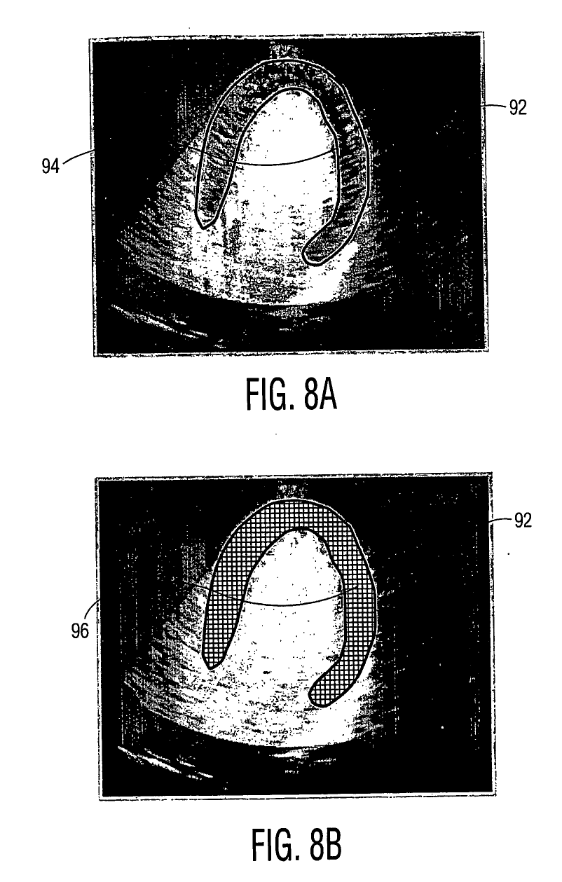

FIGS. 8a and 8b illustrate the masking of a region of interest.

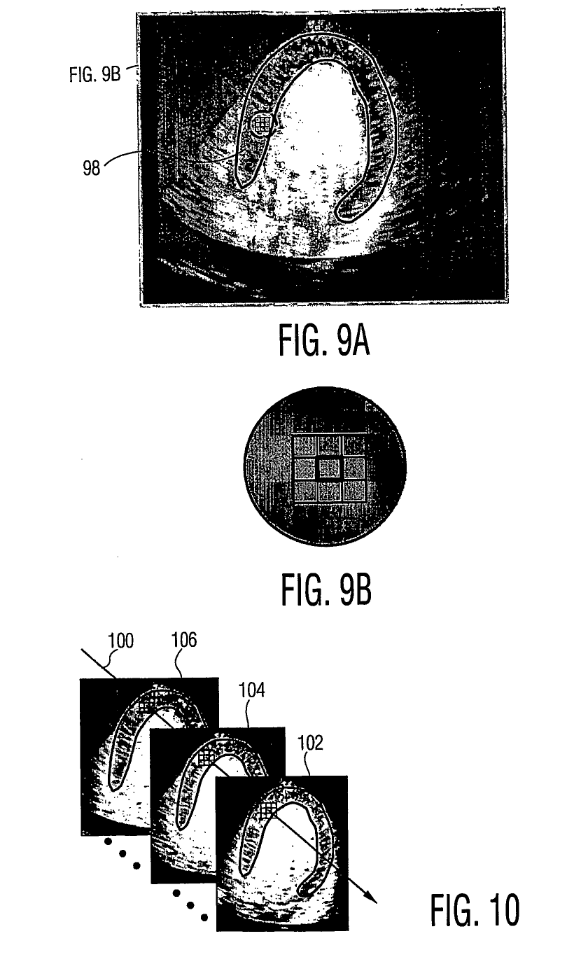

FIGS. 9a and 9b illustrate a preferred technique for quantifying pixel values in a region of interest.

FIG. 10 illustrates the selection of pixel values from a plurality of images for the determination of a perfusion curve for the pixel location.

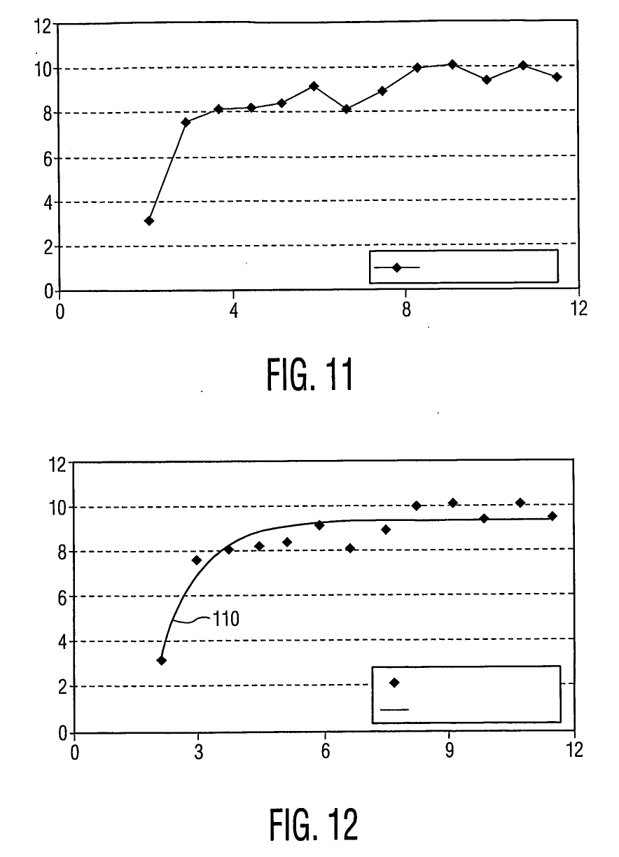

FIG. 11 illustrates the plotting of a perfusion curve from image data.

FIG. 12 illustrates the fitting of a smooth curve to the perfusion curve of FIG. 11.

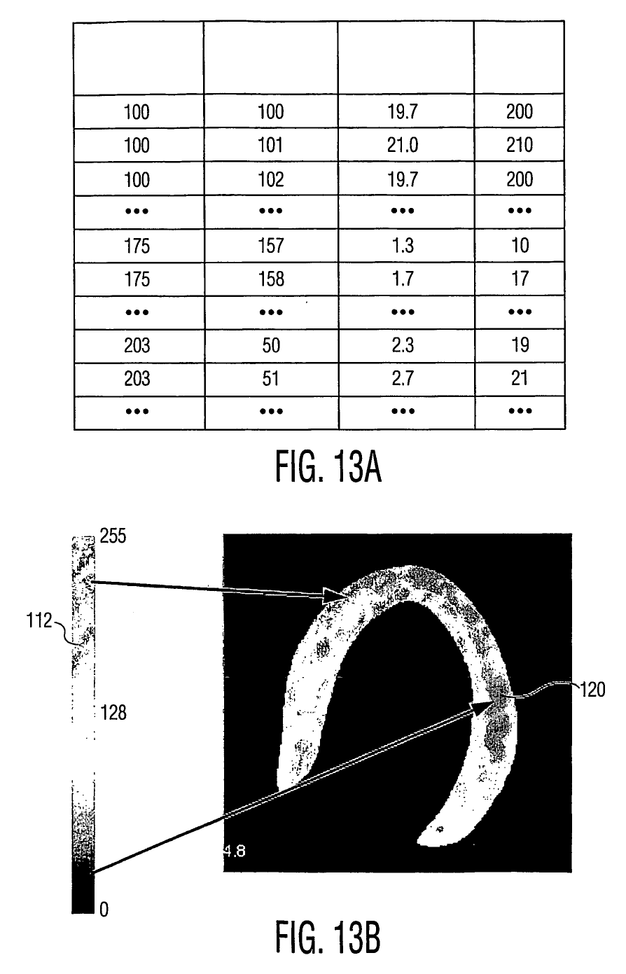

FIGS. 13a and 13b illustrate the mapping of perfusion parameters to a color scale and a two dimensional image.

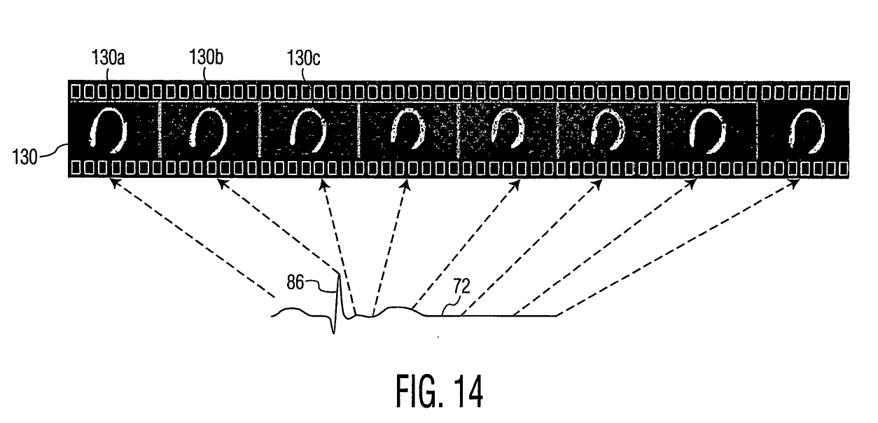

FIG. 14 illustrates a real time display of parametric perfusion images corresponding to different phases of a heart cycle.

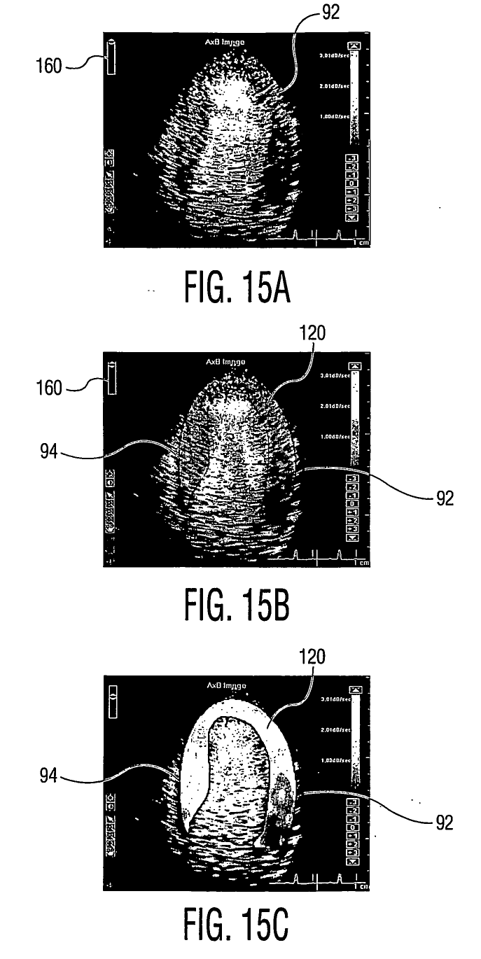

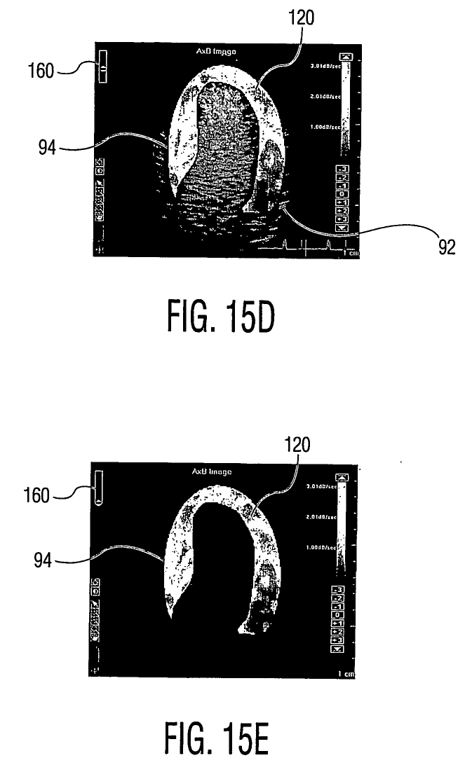

FIGS. 15a-15e illustrate a parametric perfusion image and an anatomical power Doppler image in registration with variable opacities of the two images.

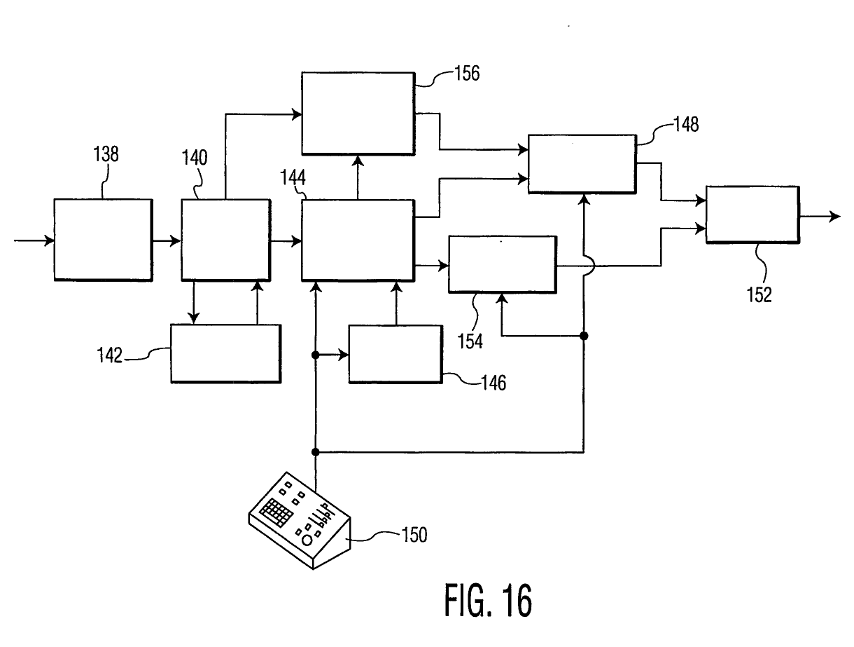

FIG. 16 is a block diagram of a portion of an ultrasonic imaging system relating to perfusion imaging in accordance with an embodiment of the invention.

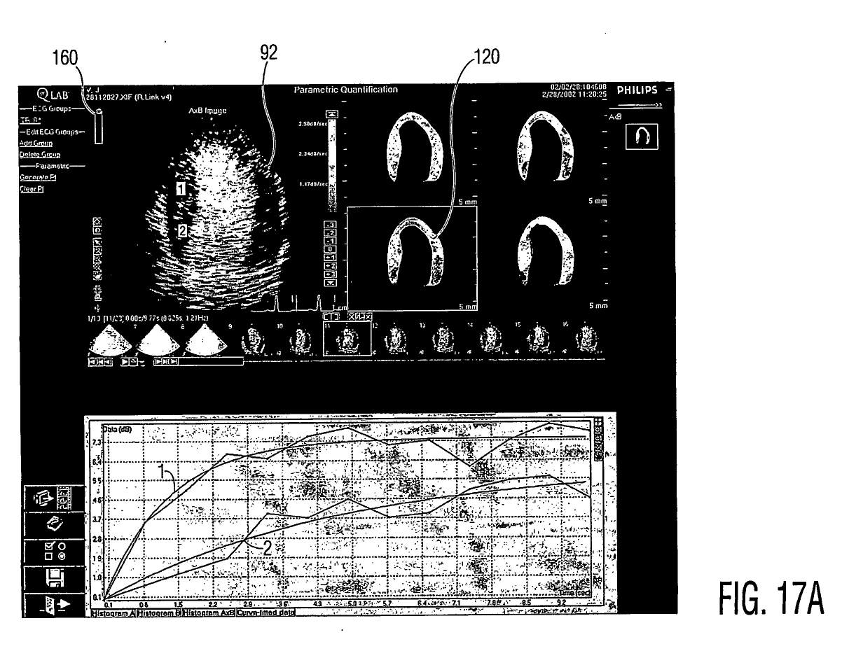

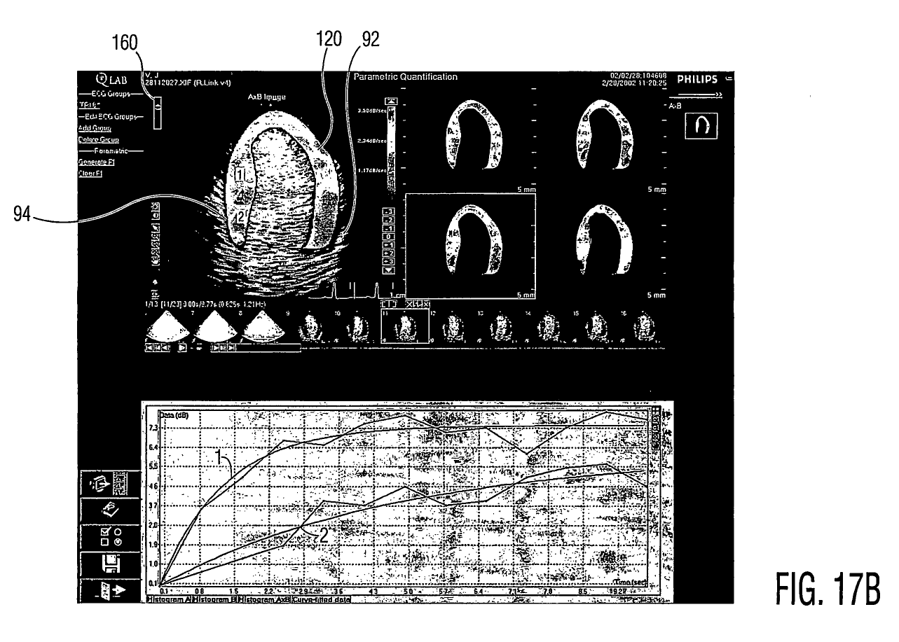

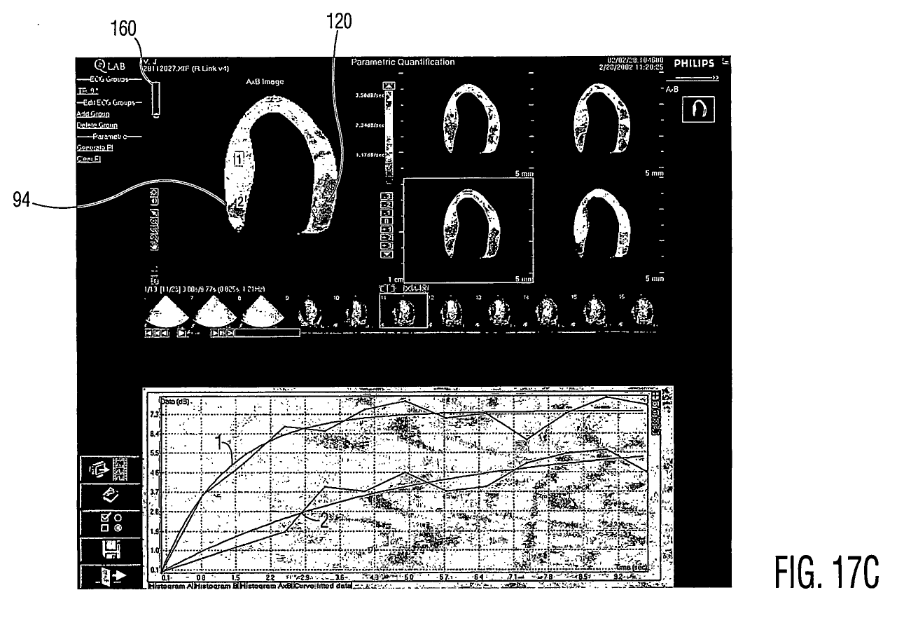

FIGS. 17a-17c are screen shots of an ultrasound system display operated in accordance with the principles of the present invention.

An ultrasonic diagnostic imaging system 10 constructed in accordance with the principles of the present invention is shown in FIG. 1. An ultrasonic scanhead 12 includes an array 14 of ultrasonic transducers that transmit and receive ultrasonic pulses. The array may be a one dimensional linear or curved array for two dimensional imaging, or may be a two dimensional matrix of transducer elements for electronic beam steering in three dimensions. The ultrasonic transducers in the array 14 transmit ultrasonic energy and receive echoes returned in response to this transmission. A transmit frequency control circuit 20 controls the transmission of ultrasonic energy at a desired frequency or band of frequencies through a transmit/receive (“T/R”) switch 22 coupled to the ultrasonic transducers in the array 14. The times at which the transducer array is activated to transmit signals may be synchronized to an internal system clock (not shown), or may be synchronized to a bodily function such as the heart cycle, for which a heart cycle waveform is provided by an ECG device 26. When the heartbeat is at the desired phase of its cycle as determined by the waveform provided by ECG device 26, the scanhead is commanded to acquire an ultrasonic image. The ultrasonic energy transmitted by the scanhead 12 can be relatively high energy (high mechanical index or MI) which destroys or disrupts contrast agent in the image field, or it can be relatively low energy which enables the return of echoes from the contrast agent without substantially disrupting it. The frequency and bandwidth of the ultrasonic energy generated by the transmit frequency control circuit 20 is controlled by a control signal ft generated by a central controller 28.

Echoes from the transmitted ultrasonic energy are received by the transducers in the array 14, which generate echo signals that are coupled through the T/R switch 22 and digitized by analog to digital (“A/D”) converters 30 when the system uses a digital beamformer. Analog beamformers may also be used. The A/D converters 30 sample the received echo signals at a sampling frequency controlled by a signal fₛ generated by the central controller 28. The desired sampling rate dictated by sampling theory is at least twice the highest frequency of the received passband, and might be on the order of at least 30-40 MHz. Sampling rates higher than the minimum requirement are also desirable.

The echo signal samples from the individual transducers in the array 14 are delayed and summed by a beamformer 32 to form coherent echo signals. The digital coherent echo signals are then filtered by a digital filter 34. In this embodiment, the transmit frequency and the receiver frequency are individually controlled so that the beamformer 32 is free to receive a band of frequencies which is different from that of the transmitted band. The digital filter 34 bandpass filters the signals, and can also shift the frequency band to a lower or baseband frequency range. The digital filter could be a filter of the type disclosed in U.S. Pat. No. 5,833,613.

Filtered echo signals from tissue are coupled from the digital filter 34 to a B mode processor 36 for conventional B mode processing. The B mode image may also be created from microbubble echoes returning in response to nondestructive ultrasonic imaging pulses. As discussed above, pulses of low amplitude, high frequency, and short burst duration will generally not destroy the microbubbles.

Filtered echo signals of a contrast agent, such as microbubbles, are coupled to a contrast signal processor 38. The contrast signal processor 38 preferably separates echoes returned from harmonic contrast agents by the pulse inversion technique, in which echoes resulting from the transmission of multiple pulses to an image location are combined to cancel fundamental signal components and enhance harmonic components. A preferred pulse inversion technique is described in U.S. Pat. No. 6,186,950, for instance, which is hereby incorporated by reference. The detection and imaging of harmonic contrast signals at low MI is described in U.S. Pat. No. 6,171,246, the contents of which is also incorporated herein by reference.

The filtered echo signals from the digital filter 34 are also coupled to a Doppler processor 40 for conventional Doppler processing to produce velocity and power Doppler signals. The outputs of these processors may be displayed as planar images, and are also coupled to a 3D image rendering processor 42 for the rendering of three dimensional images, which are stored in a 3D image memory 44. Three dimensional rendering may be performed as described in U.S. Pat. No. 5,720,291, and in U.S. Pat. Nos. 5,474,073 and 5,485,842, all of which are incorporated herein by reference.

The signals from the contrast signal processor 38, the processors 36 and 40, and the three dimensional image signals from the 3D image memory 44 are coupled to a Cineloop® memory 48, which stores image data for each of a large number of ultrasonic images. The image data are preferably stored in the Cineloop memory 48 in sets, with each set of image data corresponding to an image obtained at a respective time. The sets of image data for images obtained at the same time during each of a plurality of heartbeats are preferably stored in the Cineloop memory 48 in the same way. The image data in a group can be used to display a parametric image showing tissue perfusion at a respective time during the heartbeat. The groups of image data stored in the Cineloop memory 48 are coupled to a video processor 50, which generates corresponding video signals for presentation on a display 52. The video processor 50 preferably includes persistence processing, whereby momentary intensity peaks of detected contrast agents can be sustained in the image, such as described in U.S. Pat. No. 5,215,094, which is also incorporated herein by reference.

The manner in which perfusion can be displayed in a parametric image will now be explained beginning with reference to FIG. 2. An image 60 is obtained from a region of interest, preferably with the aid of the microbubbles used as contrast agents, as shown in FIG. 2. The anatomy shown in FIG. 2 is the left ventricle 62 of a heart, although it will be understood that the region of interest can encompass other tissues or organs. The left ventricle 62 is surrounded by the myocardium 64, which has inner and outer borders, 66, 68, respectively, that defines as an area of interest, the perfused myocardium 64. The myocardium can be distinguished for analysis by segmentation either manually or automatically using conventional or hereinafter developed techniques, as described below.

FIG. 3 illustrates a real time sequence 70 of images of the myocardium which have been acquired with a contrast agent present in the heart. The image frames in the sequence are numbered F:1, F:2, F:3, and so on. The sequence is shown in time correspondence to an ECG waveform 72 of the heart cycle. It will be appreciated that during a heart cycle 10, 20, 30, 40 or more images may be acquired, depending upon the heart rate and the ultrasound system frame rate. In one embodiment of the present invention the acquired sequence 70 of images is stored in the Cineloop memory 48. In this embodiment, during one interval 74 of images, high Ml pulses are used to acquire the images. This is typically an interval of 1-10 image frames. The use of the high intensity transmit pulses substantially disrupts or destroys the microbubbles in the image plane or volume. In this discussion these high MI frames are referred to as “flash” frames. At the end of this interval 74 low MI pulses are used to image subsequent image frames over several cardiac cycles delineated by interval 76 as the contrast agent re-perfuses the myocardium. The sequence of images shows the dynamics of the cardiac “cycle as well as contrast replenishment over many heart cycles.

Instead of acquiring a continual real time sequence of images, images can be selected out of a real time sequence or acquired at specific times in the cardiac cycle. FIG. 4 illustrates this triggered acquisition, in which the arrows 78 indicate times triggered from the ECG waveform 72 at which images are acquired at a specific phase of the heart cycle. The arrow 80 indicates the time when one or more flash frames are transmitted, followed by an interval 76 during which low MI images are acquired. In this example only one image is acquired and stored in Cineloop memory during each cardiac cycle. The user sets the trigger timing to determine which phase of the cardiac cycle to capture with the triggered images. When these images are replayed from Cineloop memory in real time, they do not show the dynamics of the cardiac cycle, as the heart is at the same phase of the cardiac cycle during each image. The sequence does show contrast replenishment in the triggered images acquired during the low MI interval 76. From image to image the viewer can see the buildup of blood in the myocardial tissue as each beat of the heart sends more blood with microbubbles into the myocardial tissue. From a time immediately following the flash frame re-perfusion can be visually observed as the myocardium becomes brighter with more microbubbles infused with each heartbeat. Tissue which does not light up as rapidly as, or to a lesser final level than, neighboring tissue can indicate the possibility of a pathological condition such as an arterial obstruction or other defect.

FIGS. 5 and 6a, 6b, and 6c illustrate the assembly of multiple single-phase sequences from a real time continuous acquisition sequence. FIG. 5 illustrates the continuous real time sequence as was shown previously in FIG. 3. The dashed lines 82 represent the divisions between different heart cycles. The illustrated images are low ML images which have been preceded by one or more flash frames (not shown). Circles 84a indicate the time of acquisition triggered by the ECG waveform 72; the image in these circles are seen to be coincident with the QRS waveform 86. These triggered images are assembled in a sequence of images at this phase of the heart, as shown by image sequence 84a in FIG. 6a. In a similar manner, triggered images are selected from the real time sequence at other phases of the heart cycle as shown by circles 84b and 84c. These triggered images are assembled into other sequences of images of their respective heart phases as shown in FIGS. 6b and 6c. This triggering may be done in real time, or in a post-processing operation in which the real time sequence of FIG. 5 is captured in Cineloop memory and the triggered sequences of FIGS. 6a, 6b, and 6c are subsequently assembled from the stored real time sequence.

The area of interest in the image, in this example the myocardium, may optionally be delineated by assisted border detection as shown in FIGS. 7a-7d. FIG. 7a illustrates a contrast image sequence 90 which may be a real time sequence 70 or a triggered sequence 80. From the image sequence 90 the user selects an image 92 which shows relatively well defined endocardial and epicardial borders. This image 92 is shown enlarged in FIG. 7b. The selected image is then processed by assisted border detection, as described in U.S. Pat. No. 6,491,636, entitled “Automated Border Detection in Ultrasonic Diagnostic Images,” the contents of which is hereby incorporated by reference. Automated or assisted border detection acts to delineate the myocardium with a border 94 as shown in FIGS. 7c and 8a. The border outline 94 on the selected image is then used to automatically delineate the border on other images in the sequence 90, as explained in the '636 patent and shown in FIG. 5d. Alternatively, the borders may be drawn on the other images in the sequence by processing them individually with the automated border detection algorithm. The area of interest where perfusion is to be represented parametrically is now clearly defined for subsequent processing. If desired, the area of interest may be further defined by a mask 96, as shown in FIG. 8b, in which the area within the border trace is masked. All pixels under the mask are to be processed in this example, while pixels outside of the mask are not processed parametrically. An assisted border detection technique is described in concurrently filed patent application serial number [attorney docket ATL-349], entitled ULTRASONIC DIAGNOSTIC IMAGING SYSTEM WITH ASSISTED BORDER TRACING,” the contents of which are herein incorporated by reference.

FIGS. 9a and 9b illustrate a preferred technique for processing the pixels within a region of interest, in this case; the myocardium delineated by the border tracing 94 in FIG. 8a. As FIGS. 9a and 9b show, for each pixel within the region of interest a mean image intensity value is calculated for a pixel and its surrounding eight neighboring pixels. Pixel values are calculated in this manner for each pixel in the myocardium 98 in this example, and the process is repeated for every pixel in the same location for each image in the sequence as shown for images 102, 104, 106 in FIG. 10. The common location pixel values are, at least conceptually, then plotted graphically as a function of time and mean intensity as shown in FIG. 11, which shows a plot of the common location pixel values intersected by arrow 100 in FIG. 10. The common location pixels are then used to develop a perfusion parameter for display in a two- or three-dimensional image of the region of interest. In a preferred embodiment, parameters are produced by fitting the plotted values to a curve 110 of the form:
I(t)=A(1−exp^{(-B*t)})+C
where A is the final curve intensity, B is proportional to the initial slope of the curve, and C is a floating constant. A drawn curve 110 of this form is illustrated in FIG. 12. Parameters may then be formed using the values A, B, and combinations thereof (A*B, A/B, etc.) as shown below.

FIG. 13a-13b illustrate the creation of a parametric image from a parameter value of the form A*B using the curve characteristics described above. In the table of FIG. 13a, the first two columns indicate the locational coordinates of pixels in a two dimensional image. For three dimensional images a third coordinate will be used. The A*B parameter value for each pixel location is represented in the third column. The range of parameter values, represented by the color bar 112 calibrated from zero to 255 between FIGS. 13a and 13b, is then used to encode (map) each parameter value to a color, brightness, or other display characteristic. The colors are then displayed in their respective locations in a two or three dimensional parametric image 120, as shown in FIG. 13b, in which the perfusion of the myocardium of the heart is parametrically displayed.

The techniques of the present invention may be used to produce a single static image 120 as shown in FIG. 13b, or they may be used to produce a sequence of parametric images which may be displayed in sequence or in real time. For instance, FIG. 14 illustrates a sequence of parametric perfusion images from different phases of the heart cycle, as indicated by the arrows drawn from the different points on the ECG waveform 72 to individual images in the sequence 130. Each parametric image in the parametric image sequence 130 can be formed by a different one of the different phase sequences of FIGS. 6a, 6b, and 6c, for instance. The images of FIG. 6a are used to produce parametric image 130a, the images of FIG. 6b are used to produce parametric image 130b, and the images of FIG. 6c are used to produce parametric image 130c. When sequence 130 is played in full or partial real time, it will reveal the changing perfusion in the various locations in the myocardium during the different phases of the heart cycle. This image sequence thus reveals both myocardial perfusion information and the dynamics of wall motion of the endocardium in the same diagnostic image sequence.

A method of displaying a parametric image in combination with the anatomy on which the parametric image is based is shown in FIGS. 15a-15e. FIG. 15a shows an ultrasound image display with an ultrasonic power Doppler image 92 of the left ventricle of a heart containing a contrast agent. The bright center of the image is the cavity of the left ventricle which contains a substantial amount of contrast agent and the darker surrounding area is the myocardium which is just beginning to be perfused with blood containing the contrast agent. At the upper left corner of the display is a rectangular box 160 containing a white slider at the top of the box. When the slider in the box 160 is at the top in this embodiment, the anatomical image 92 is fully opaque and a corresponding parametric image overlay is fully transparent. In FIG. 15b the white slider of the box 160 has been moved by user manipulation of a pointing device such as a mouse or trackball to a slightly lower position in the box 160. In this slider position the structural image 92 is still opaque but the parametric image overlay 120 is now translucent over the myocardium for which it displays perfusion. The border tracing 94 around the myocardial tissue is also visible in this image display. In FIG. 15c the slider has been moved to approximately the center of the box 160, causing the parametric image overlay 120 to be fully opaque over the myocardium of the structural image 92. The tissue and blood flow in the myocardium is now fully obscured by the parametric image overlay 120. In a constructed embodiment the values of the perfusion parameters of the parametric image are mapped to the colors of the color bar shown at the upper right of the display using a map known as a “stoplight” map. In a stoplight map, areas of the myocardium exhibiting normal or satisfactory blood flow perfusion are shown in green, which is shown as a grey shading in FIG. 15c. Areas of the myocardium exhibiting questionable or suspect perfusion are shown in yellow, which appears as the lightest shading in the parametric image of FIG. 15c, and areas of the myocardium exhibiting poor or no perfusion are shown in red, which appears as the darkest shade in the parametric image. Other color maps, such as different shades or brightnesses of a single color, may also be used at the preference of the user.

In FIG. 15d the slider is moved to a lower position in the box 160, causing the structural image 92 to become slightly transparent or translucent. Finally, when the slider is moved to the bottom of the box 160, the structural image become fully transparent, leaving only the opaque parametric image 120 as shown in FIG. 15e. Thus by manipulating the slider the clinician is able to easily view the perfusion of the myocardial tissue, the blood flow itself, or both simultaneously simply by moving the slider 160 and varying the relative opacity of the structural image 92 and the parametric image 120. The two images remain in anatomical registration so that the clinician can always relate the perfusion to the point in the body where the perfusion exists and is being measured.

The portion of an ultrasound system which enables this opacity control is shown in FIG. 16. Echo signals are received by a harmonic signal detector 138 which separates and detects harmonic signal components from echo signals returned by tissue and/or contrast agent in the blood flow. Harmonic signal separation can be performed by bandpass filtering or by pulse inversion as described in U.S. Pat. No. 5,706,819 (Hwang), U.S. Pat. No. 5,951,478 (Hwang et al.), and U.S. Pat. No. 6,193,662 (Hwang). The harmonic signals are detected by amplitude detection or Doppler processing (see U.S. Pat. No. 6,095,980) and stored in an image data memory 140. The image data used for an image is forwarded to a scan converter 142 which produces image data of the desired image format, e.g., sector, rectangular, virtual apex, or curved linear. The scan converted image data is stored in the image data memory from which it is accessed by an assisted border detector 144 and a perfusion parameter processor 156. The assisted border detector 144 is responsive to input from a trackball pointing device on a user control panel 150 to locate control points with reference to the image data and position and stretch boundary templates with respect to the image data, as discussed more fully in the concurrently filed patent application serial number [attorney docket ATL-349]. The template data is provided by a border template storage device 146. As the control points and borders are being drawn and positioned on the image, the control point and border data produced by the assisted border detector 144 is applied to a graphics processor 148, which produces a graphic overlay of the control points and border to be displayed with the image data. The delineated border is also provided to the perfusion parameter processor 156, which computes and color maps perfusion parameters over the area or volume delineated by the border, as explained above in conjunction with FIGS. 8-13. The perfusion color values for the region of interest are also coupled to the graphics processor 148 which combines the perfusion parameters with the border to form the parametric image as shown in FIGS. 15b-15e, and also adds the graphic of the slider 160. The image data corresponding to (and therefore in anatomical registration with) the parametric image is coupled to an image data processor 154. Signals from the pointing device on the user control panel used to move the slider 160 are coupled to both the image data processor 154 and the graphics processor 148, where the signals are used to appropriately adjust the relative opacities of the structural image of the image data processor and the parametric image of the graphics processor. The graphic overlay of the slider and parametric image and the structural image data are stored in a display memory 152, from which they are accessed for display by the video processor 50.

FIGS. 17a-17c are reproductions of an ultrasound display which show parametric images, an image sequence, graphical plots of perfusion and a combined structural and parametric image in accordance with the principles of the present invention. In the center of the display is a horizontal strip of sequential images of a Cineloop of images which are used to calculate perfusion parameters. The first three image frames of the strip show flash frames during which the contrast agent is disrupted or destroyed prior to the perfusion measurement. The image frame highlighted by the bright outline in the center of the strip is shown in enlarged form as structural image 92 in the upper left part of the display. To the right of the large structural image of the left ventricle are four parametric images illustrating perfusion by different parameters. The upper left parametric image shows the final perfusion level attained in the myocardium, a representation of steady-state maximum perfusion. This would be the final plateau or amplitude of the curve 110 in FIG. 12, for instance. The upper right parametric image shows the time constant of the perfusion curve, or perfusion rate. This would be the time constant (slope) of the curve 110, for instance. The lower left parametric image illustrates the A×B perfusion, as explained above with reference to the equation for the curve 110. This parametric image is highlighted by a bright outline, indicating that this parametric image 120 will be shown in registration with the large structural image 92 to the left. The lower right parametric image shows the quality of the curve fits to the perfusion data of the image sequence, which provides an indication of the reliability of the acquired data for perfusion analysis.

The large structural image 92 is seen to have two white markers located on the myocardium and denotes as “1” and “2”. The perfusion curves for these two points of the myocardium, calculated by the same process used to produce the highlighted parametric image 120, are shown at the bottom of the display. One or more perfusion curves may be displayed concurrently in this area of the display. Each perfusion curve is shown two way: as perfusion data points connected by line segments such as that shown in FIG. 11, and as the curve fit to those data points such as curve 110 of FIG. 12. The user can click on any point of the anatomy of the image 92 and immediately see the perfusion data and curve for the designated point in the body.

In the display of FIG. 17a the slider of the box 160 is seen to be positioned at the top of the box, causing the structural image to be fully opaque and the parametric image to be fully transparent. When the user moves the slider lower in the box 160 as shown in FIG. 17b, the parametric image in registration with the structural image begins to appear. In this illustration the opaque parametric image 120 is shown over the opaque anatomical image 92 of the heart. In FIG. 17c the slider has been moved to the bottom of the box 160 and the anatomical image 92 has become completely transparent while the parametric image remains fully opaque. The embodiment of FIGS. 17a-17c provides the clinician with a wide array of diagnostic images and data displays for a rapid and accurate diagnosis of cardiac and other conditions.

It will be appreciated that the variable opacity control may find utility whenever an image depicting an anatomical parameter is shown in registration with an image of the anatomy from which the parameter is calculated. For example, anatomical Doppler images such as color flow images show the anatomy of the heart or a vessel with a color overlay of a parameter of the anatomy such as the velocity of flow of the blood in the vessel or organ. The variable opacity control of the present invention could be used with these images to simultaneously show both the flowing blood and its velocity in anatomical registration, with the blood or the velocity parameter either wholly opaque, transparent, or translucent.

It will also be appreciated that, while a continuously variable slider is shown in the previous embodiments, an incremental stepped control may also be employed, in which the relative opacity of the anatomical and parametric images are adjusted from one discrete relative opacity setting to another.

It will be readily apparent to those skilled in the art that instead of using a single slider for control of the opacity of both the parametric and the B mode image, the opacity control function can be partitioned among two or more separate sliders. For example, one slider could be used to control the opacity of the anatomical display while a second slider is used to control the opacity of the parametric overlay. It will also be apparent that the relative opacity of the two displays can be adjusted dynamically while the anatomy and perfusion images are played as a real time image sequence.

1. A method of simultaneously displaying a parametric diagnostic image and an anatomical diagnostic image of the region of interest corresponding to the parametric diagnostic image, comprising:
acquiring an anatomical image of a region of interest of a subject;
acquiring a parametric image of the region of interest of the subject; and
displaying the parametric image in anatomical registration with the anatomical image, wherein the relative opacity of the registered parametric image and anatomical image is variable.

2. The method of claim 1, wherein acquiring an anatomical image comprises acquiring an anatomical image of a region of the body containing blood flow; and
wherein acquiring a parametric image comprises acquiring a parametric image of a characteristic of blood flow in the region of the body.

3. The method of claim 2, wherein acquiring a parametric image comprises acquiring a parametric image of the blood flow perfusion of the tissue in the region of the body.

4. The method of claim 3, further comprising directing a flow of contrast agent to the region of interest of the subject.

5. The method of claim 1, further comprising varying the relative opacity of the registered parametric image and anatomical image in a continuous manner.

6. The method of claim 1, further comprising varying the relative opacity of the registered parametric image and anatomical image in a stepwise manner.

7. The method of claim 5, wherein varying the relative opacity further comprises varying the opacity within a range extending from an opaque anatomical image and a transparent parametric image; to an opaque anatomical image overlaid with an opaque parametric image; to a transparent anatomical image and an opaque parametric image.

8. The method of claim 7, wherein varying the opacity within a range further comprises varying the opacity within a range which includes an opacity setting in which a translucent parametric image is shown in registration with a substantially opaque anatomical image.

9. The method of claim 6, wherein varying the relative opacity further comprises varying the opacity within a range extending from an opaque anatomical image and a transparent parametric image; to an opaque anatomical image overlaid with an opaque parametric image; to a transparent anatomical image and an opaque parametric image.

10. A diagnostic imaging system for displaying a parametric image in anatomical registration with an anatomical image of a region of interest of a subject comprising:
a source of diagnostic images of a region of interest of a subject;
a source of parametric images of the region of interest of the subject;
a display coupled to the source of diagnostic images and the source of parametric images which displays a diagnostic image and a corresponding parametric image in anatomical registration;
a display processor coupled to the display which acts to set the relative opacity of the registered diagnostic image and parametric image; and
a user control, coupled to the display processor, by which a user can set the relative opacity of the registered diagnostic image and parametric image.

11. The diagnostic imaging system of claim 10, wherein the source of diagnostic images comprises a source of diagnostic images of a region of interest containing blood flow; and wherein the source of parametric images comprises a source of at least one parametric image of a characteristic of the blood flow in the region of interest.

12. The diagnostic imaging system of claim 11, wherein the source of parametric images comprises a source of at least one parametric image of the blood flow perfusion in the tissue depicted in the region of interest.

13. The diagnostic imaging system of claim 10, wherein the display processor further comprises an opacity processor which acts to set the relative opacity of the registered diagnostic image and parametric image within a range varying from an opaque diagnostic image and a transparent parametric image; to an opaque diagnostic image overlaid with an opaque parametric image; to a transparent diagnostic image and an opaque parametric image.

14. The diagnostic imaging system of claim 10 wherein the user control comprises a user control, coupled to the display processor, by which a user can set the relative opacity of the registered diagnostic image and parametric image within a continuous range of relative opacity settings.

15. The diagnostic imaging system of claim 10 wherein the user control comprises a user control, coupled to the display processor, by which a user can set the relative opacity of the registered diagnostic image and parametric image to one of a discrete number of relative opacity settings.

16. The diagnostic imaging system of claim 10 wherein the user control comprises a user control, coupled to the display processor, by which a user can set the relative opacity of the registered diagnostic image and parametric image to a setting in which the display displays a translucent parametric image in registration with a substantially opaque diagnostic image.

17. The diagnostic imaging system of claim 10 wherein the display further comprises a display which displays in real time a diagnostic image sequence and a corresponding parametric image sequence in anatomical registration.

18. The diagnostic imaging system of claim 10 wherein the user control comprises a user control, coupled to the display processor, by which a user can set the relative opacity of the registered diagnostic image and parametric image to a setting in which the display displays a translucent diagnostic image in registration with a substantially opaque parametric image.

19. The diagnostic imaging system of claim 10 wherein the user control further comprises a plurality of separate user controls by which a user can set the opacity of the parametric image and the opacity of the registered diagnostic image.
